(19) United States
(12) Patent Application Publication
(10) Pub. No.: US 2025/0120807 A1
(43) Pub. Date: Apr. 17, 2025

(54) VENTRICLE ACCESS PIGTAIL DEVICE

(71) Applicant: BOSTON SCIENTIFIC SCIMED, INC., Maple Grove, MN (US)

(72) Inventors: Donal Kyne, Galway (IE); Tim O'Connor, Galway (IE); Chris Cullen, Galway (IE)

(73) Assignee: BOSTON SCIENTIFIC SCIMED, INC., Maple Grove, MN (US)

(21) Appl. No.: 18/911,711

(22) Filed: Oct. 10, 2024

Related U.S. Application Data
(60) Provisional application No. 63/543,615, filed on Oct. 11, 2023.

(51) Int. Cl. A61F 2/24

(52) U.S. Cl. CPC A61F 2/2418; A61F 2/243; A61F 2230/0006

(57) ABSTRACT

A system for use in replacement heart-valve implant procedures, the system including a guidewire and an elongate member. The elongate member having a side port disposed proximal of a distal tip of the elongate member. The guidewire slideably disposed within a first lumen of the elongate member and configured to be advanced out the side port proximate a leaflet of a native heart valve of a patient. The side port includes an angled wall on the distal end of the port configured such that when the guidewire is advanced out of the side port, it extends out at an angle relative to the elongate member.

### CROSS-REFERENCE TO RELATED APPLICATIONS

This application claims the benefit of priority of U.S. Provisional Application No. 63/543,615 filed Oct. 11, 2023, the entire disclosure of which is hereby incorporated by reference.

### TECHNICAL FIELD

The disclosure pertains to medical devices and more particularly to delivery systems for replacement heart valves, and methods for using such medical devices and systems.

### BACKGROUND

A wide variety of medical devices have been developed for medical use including, for example, medical devices utilized to replace heart valves. Heart function can be significantly impaired when a heart valve is not functioning properly. When the heart valve is unable to close properly, the blood within a heart chamber can regurgitate, or leak backwards through the valve. Valve regurgitation may be treated by replacing or repairing a diseased valve, such as an aortic valve. Surgical valve replacement is one method for treating the diseased valve, however this requires invasive surgical openings into the chest cavity and arresting of the patient's heart and cardiopulmonary bypass. Minimally invasive methods of treatment, such as transcatheter aortic valve implantation (TAVI) or transcatheter aortic valve replacement (TAVR), generally involve the use of delivery catheters that are delivered through arterial passageways or other anatomical routes into the heart to replace the diseased valve with an implantable prosthetic heart valve.

In most cases, accessing a heart valve for replacement requires placement of a guidewire through the valve leaflets. Of the known delivery devices and methods for accessing a heart valve for replacement, each has certain advantages and disadvantages. There is an ongoing need to provide alternative devices and methods for manufacturing and using the medical devices.

### SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter. An example medical device includes a guidewire, and an elongate member including a first lumen having a side port disposed proximal of a distal tip of the elongate member, wherein the guidewire is slideably disposed within the first lumen and configured to be advanced out the side port proximate a leaflet of a native heart valve of a patient, wherein the side port includes an angled wall defining a distal end of the first lumen, wherein the angled wall of the side port is configured such that when the guidewire is advanced out of the side port, the guidewire extends out at an angle relative to the elongate member.

Alternatively or additionally to the embodiment above, the elongate member is a pigtail catheter.

Alternatively or additionally to any of the embodiments above, the pigtail catheter includes a preformed recurve portion beginning at the distal tip of the pigtail catheter and extending proximally in an unconstrained configuration.

Alternatively or additionally to any of the embodiments above, the preformed recurve portion is sized and shaped to fit within a non-coronary cusp of the patient's valve.

Alternatively or additionally to any of the embodiments above, the angled wall of the side port is configured to direct the guidewire between the right coronary cusp and left coronary cusp of the native heart valve.

Alternatively or additionally to any of the embodiments above, the angled wall of the side port is angled 90 degrees to 120 degrees relative to an inner wall defining the first lumen in a region of the side port.

Alternatively or additionally to any of the embodiments above, the side port is spaced at least 40 mm proximally from a distal end of the preformed recurve portion of the pigtail catheter.

Alternatively or additionally to any of the embodiments above, the elongate member is a catheter that includes the first lumen and a second lumen.

Alternatively or additionally to any of the embodiments above, the device may further include a pigtail catheter slideably disposed within the second lumen of the catheter.

Alternatively or additionally to any of the embodiments above, the pigtail catheter includes a preformed recurve portion beginning at a distal tip of the pigtail catheter and extending proximally in an unconstrained configuration.

An example method of accessing a valve for implanting a replacement heart-valve includes inserting a distal tip of a stent-valve delivery system through a patient's aorta to a position upstream of the aortic valve, the delivery system including an elongate member including a first lumen having a side port disposed proximal of a distal tip of the elongate member, and a guidewire slideably disposed within the first lumen of the elongate member, advancing the elongate member adjacent the patient's non-coronary cusp, advancing the guidewire distally out of the side port, the side port including an angled wall defining a distal end of the first lumen, wherein the angled wall of the side port directs a distal end of the guidewire between the right coronary cusp and left coronary cusp of the aortic valve and into a ventricle, and removing the elongate member, leaving the guidewire extending through the aortic valve.

Alternatively or additionally to any of the embodiments above, the elongate member is a pigtail catheter.

Alternatively or additionally to any of the embodiments above, the pigtail catheter includes a preformed recurve portion beginning at the distal tip of the pigtail catheter and extending proximally in an unconstrained configuration.

Alternatively or additionally to any of the embodiments above, after removing the elongate member the method further comprises inserting a second pigtail catheter over the guidewire and through the aortic valve into the ventricle.

Alternatively or additionally to any of the embodiments above, the method may further include inserting a transcatheter aortic valve replacement device over the second pigtail catheter.

Alternatively or additionally to any of the embodiments above, the method may further include removing the second pigtail catheter.

An example medical device includes a pigtail catheter including a side port disposed proximal of a distal tip of the pigtail catheter, and a guidewire, wherein the side port has an angled wall configured to direct the guidewire away from the pigtail catheter, wherein the guidewire is slideably disposed within a first lumen of the pigtail catheter and configured to be advanced out the side port proximate a leaflet of a native heart valve of a patient, wherein when advanced out of the side port, the guidewire extends out at an angle configured to direct the guidewire through the native heart valve, wherein the pigtail catheter includes a preformed recurve portion beginning at the distal tip of the pigtail catheter and extending proximally in an unconstrained configuration.

Alternatively or additionally to the embodiment above, the side port is disposed proximal of a base of the preformed recurve portion.

Alternatively or additionally to the embodiment above, the angled wall of the side port is angled 90 degrees to 120 degrees relative to an inner wall defining the first lumen in a region of the side port.

Alternatively or additionally to the embodiment above, the side port is spaced at least 40 mm proximally from the base of the preformed recurve portion of the pigtail catheter.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1a is a side view of an example pigtail catheter with a side port.

FIG. 1b is a cross-sectional view of a portion of the pigtail catheter of FIG. 1a.

FIG. 2a is a side view a pigtail catheter inserted through an example slideable casing.

FIG. 2b is a cross-sectional view of the pigtail catheter inserted through a slidable casing of FIG. 2a.

FIG. 3 is a cross-sectional view of a portion of a heart with a pigtail catheter in the aorta.

FIG. 4 is a top view of an aortic valve.

FIG. 5a illustrates a stent delivery system disposed within the aortic arch and aortic valve.

FIG. 5b illustrates the delivery system of FIG. 5a with the distal and proximal sheaths withdrawn from the stent valve and the stent valve fully expanded.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “withdraw”, variants thereof, and the like, may be generally considered with respect to the positioning, direction, and/or operation of various elements relative to a user/operator/manipulator of the device, wherein “proximal” and “withdraw” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc. Additionally, the term “substantially” when used in reference to two dimensions being “substantially the same” shall generally refer to a difference of less than or equal to 5%.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following description should be read with reference to the drawings, which are not necessarily to scale, wherein similar elements in different drawings are numbered the same. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

Performing transcatheter aortic valve replacement (TAVR) procedures requires accessing the ventricle with a guidewire extending through the aorta. Access can be difficult due to interaction of the guidewire with the diseased native aortic valve leaflets. This difficulty may result in increased procedural time, increased exposure to fluoroscopy and radiation for the patient, and frustration for the Physician.

A pigtail catheter is typically used during TAVR procedures to inject contrast and use as a placement indicator, as it sits in the non-coronary cusp of the native anatomy through the procedure. As described below, a modified pigtail catheter is one embodiment of a device that may be used in a system for use in replacement heart-valve implant procedure, where the pigtail catheter includes a lumen to track a guidewire that will exit the lumen at a controlled angle directing it between the native leaflets and into the ventricle when the pigtail is placed in the non-coronary cusp.

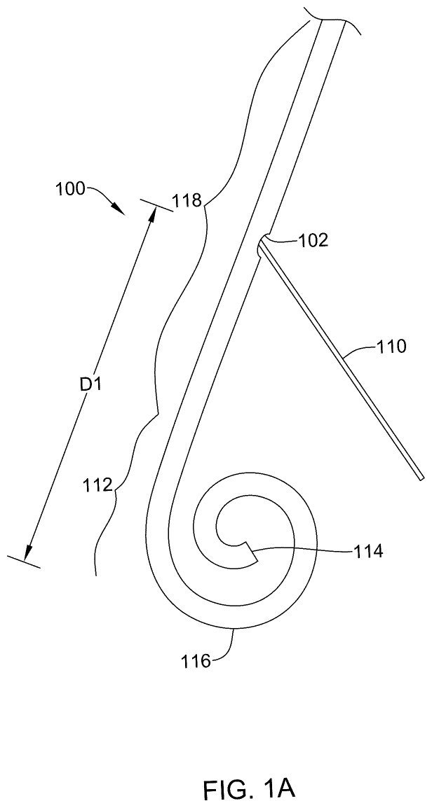

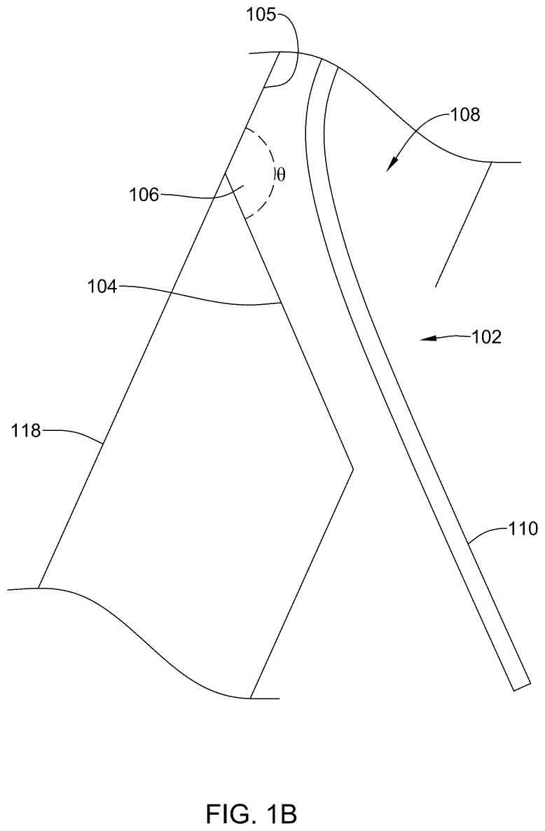

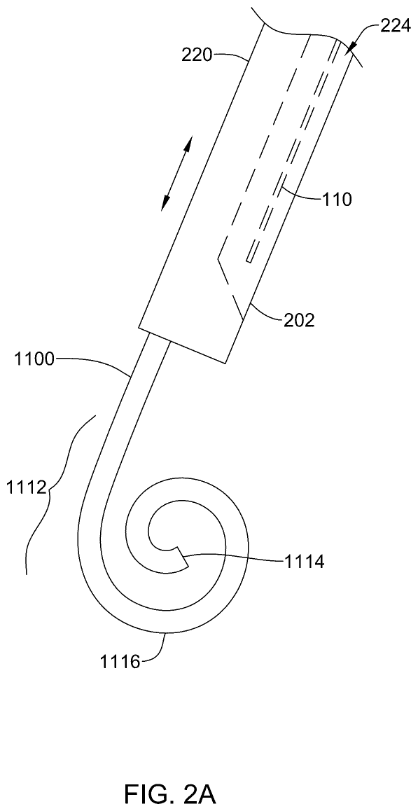

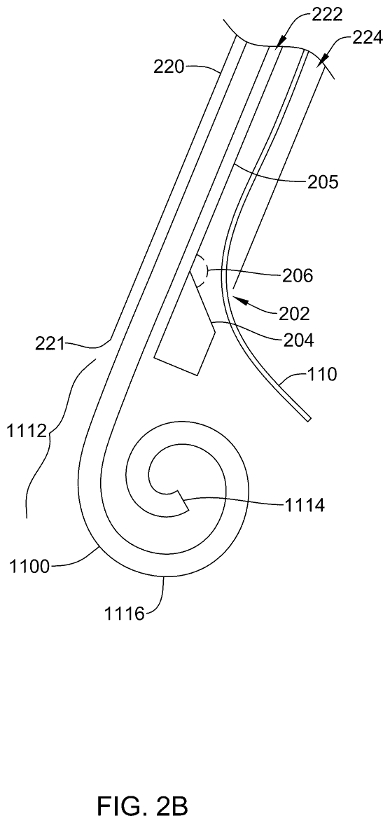

FIGS. 1A-2B illustrate different embodiments of a system for use in replacement heart-valve implant procedures, involving an elongate member with an angled side port for directing a guidewire extending therethrough toward a specific anatomical location.

In FIG. 1a the elongate member with an angled side port is a pigtail catheter 100, shown in an unconstrained configuration. The pigtail catheter 100 may include a side port 102, a distal recurve portion 112, and a straight portion 118. A guidewire 110 is shown extending through the pigtail catheter and out the side port 102. In some embodiments the straight portion 118 may be slightly curved or bent, while still being less curved than the distal recurve portion 112. The distal recurve portion may include a section of the distal end of the pigtail catheter that is spirally curved inward into a loop. The curve may be 360 degrees or more such that when in the curved, unconstrained configuration, the distal tip 114 of the pigtail catheter is more proximal than the base 116 of the recurve portion 112 which defines the distalmost extent of the pigtail catheter when in the curved, unconstrained configuration. The curve of the catheter puts the distal tip 114 within the middle of a spiral, such that the distal tip 114 is spaced apart from the walls of a body lumen within which the catheter is advanced into. The distal recurve portion 112 of the pigtail catheter may be flexible such that it can be straightened by passage of a guide wire therethrough, in order to be passed through the aorta or aortic valve. In some embodiments, the pigtail catheter may be inserted into a delivery device which straightens the distal recurve portion until reaching the desired body lumen (aorta, ventricle etc.). When delivered from the delivery device, the distal recurve portion 112 of the pigtail catheter returns to its unconstrained configuration, forming the coiled shape shown in FIG. 1a. When in the unconstrained configuration, the distal recurve portion 112 of the pigtail catheter 100 may be sized and shaped to fit within a certain body structure. For example, the distal recurve portion 112 may be sized and shaped to fit within the non-coronary cusp of a patient's heart.

The side port 102 of the pigtail catheter is located on the straight portion 118 of the pigtail catheter 100. The side port 102 may be disposed on the same side of the pigtail catheter as the spiral of the distal recurve portion 112, as shown in FIG. 1A. In other embodiments, the side port 102 may be on the opposite side from the spiral, however, in either embodiment, the side port 102 and the spiral of the distal recurve portion 112 lie in the same plane.

In some embodiments, the side port may be spaced apart a distance D1 proximally from the distal base 116 of the recurve portion 112. In some examples, the distance D1 may be 30 mm to 60 mm or 40 mm to 50 mm. In one example, the distance D1 may be at least 40 mm. The guidewire 110 is advanced through a lumen 108 of the pigtail catheter as shown in more detail in FIG. 1b. The guidewire 110 may have a curve or bend to direct the distal end of the guidewire to a predetermined location (e.g. between leaflets of an aortic valve). In some embodiments the guide wire may have a generally straight unconstrained configuration, while being flexible so as to be delivered through the anatomy to the desired treatment location. The angle at which the guidewire exits the side port 102 is dependent upon an angle of the side port as discussed in more detail below.

FIG. 1b illustrates a cross section of the straight portion 118 of a pigtail catheter 100 including the side port 102. The interior of the pigtail catheter 100 includes a lumen 108 extending proximally to the end of the catheter. A guidewire 110 may be inserted into and advanced through this lumen 108. In some embodiments, the lumen 108 may end upon reaching the side port 102, and the pigtail catheter distal of the side port 102 may be solid, as shown in FIG. 1b. In some embodiments there may be a second lumen extending from the distal tip of the catheter to the proximal end. In some embodiments the second lumen may also end upon reaching the side port 102. The side port 102 includes an opening connecting the lumen 108 of the catheter 100 to the exterior of the catheter (e.g. body lumen). The side port may also include an angled wall 104 defining the distal end of the lumen 108 and side port 102. This angled wall extends at an angle (θ) 106 from the inner wall defining the lumen 108. The angled wall 104 directs the guidewire 110 out of the side port 102 distally at an angle. The angle at which the guidewire exits the side port may correspond to the angle 106. In some embodiments the angle 106 of the angled wall 104 may be between 90 degrees and 120 degrees. The angle 106 may be measured relative to the inner wall 105 defining the first lumen 108 of the pigtail catheter in the region of the side port 102. The guidewire may be advanced distally out of the side port, and also retracted proximally back into the side port.

FIGS. 2A and 2B illustrate an alternative embodiment of an elongate member with a side port for directing a guidewire to a specific anatomical location. In this embodiment the elongate member is a dual lumen catheter 220 with a side port 202 used with a pigtail catheter 1100 having a recurve portion 112 with a distal base 116. The pigtail catheter 1100 may be devoid of any side ports. In some embodiments, it may be advantageous to adjust the height of the side port 202 relative to the distal base 11116 of the recurve portion 112 of the pigtail catheter 1100. For example, adjusting the height of the side port 202 may be needed to adjust for the unique anatomy of a patient's aortic valve. The pigtail catheter 1100 may be inserted through a first lumen 222 in the dual lumen catheter 220, as shown in FIG. 2B. The first lumen 222 may extend from the distal end 221 of the catheter 220 to the proximal end (not shown). A second lumen 224 in the catheter 220 may extend from the proximal end of the catheter to a side port 202 proximal of the distal end 221. In some embodiments, the second lumen 224 may terminate at the side port 202. The side port 202 may be spaced proximally 5 mm to 30 mm from the distal end 221 of the catheter.

The catheter 220 may be slideably disposed around and axially moveable relative to the pigtail catheter 1100 such that it can be moved in both the distal and proximal directions. The distal recurve portion 1112 of the pigtail catheter 1100 may be advanced to rest within the non-coronary cusp of the patient.

A guidewire 110 may be inserted through the second lumen 224 of the catheter, while the pigtail catheter remains in the first lumen 222. The guidewire may be advanced out the side port 202 in the distal direction and retracted proximally back into the side port. The second lumen 224 including the side port 202, may further include an angled wall 204 that directs the guide wire out of the catheter at an angle. In some embodiments the angled wall 204 may extend at an angle 206 of between 90 degrees and 120 degrees from the inner wall 205 defining the second lumen 224. The guidewire 110 may exit the side port at the angle 206 of the angled wall 204. In some embodiments, the unique anatomy of a patient may lead to the aortic valve being spaced such that the side port is further away from the target leaflet openings than expected. The height of the slidable catheter 220 may be adjusted to compensate for the distance, allowing the guidewire, when advanced, to reach the target leaflets. Adjusting the height of the catheter 220 may be achieved by moving the catheter axially relative to the pigtail catheter 1100 which sits in the non-coronary cusp. In another example, a physician may advance the guidewire distally to the leaflets of the patient's aortic valve. If the physician is unable to pass the guidewire through the leaflets, they may adjust the height of the side port relative to the distal end of the pigtail catheter by sliding the catheter 220 in the proximal or distal direction over the pigtail catheter during the procedure.

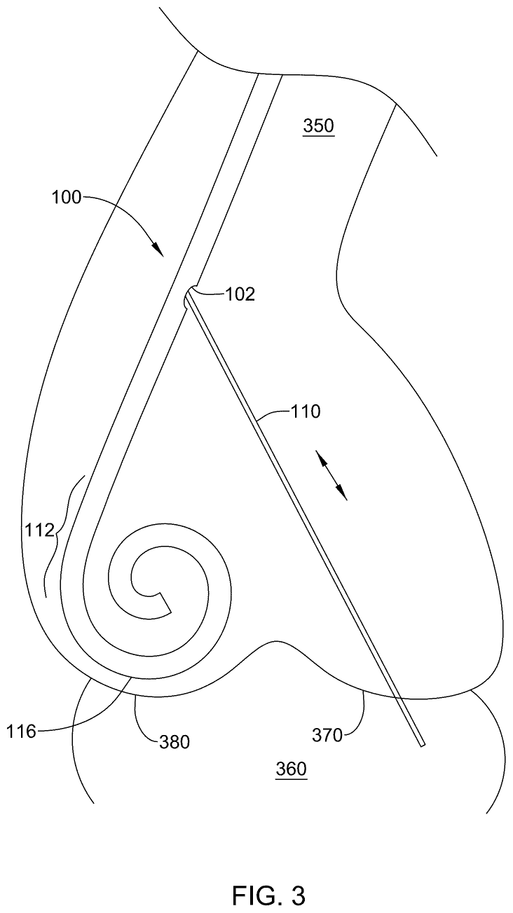

The above described systems may be used a TAVR procedure. For example, FIG. 3 illustrates an example method in which a pigtail catheter has been advanced into the non-coronary cusp 380 of a patient upstream the aortic valve. As shown in FIG. 3, the pigtail catheter advanced into the non-coronary cusp 380 adjacent the aortic valve is the pigtail catheter 100 of FIG. 1a. In other embodiments, the pigtail catheter advanced adjacent the aortic valve may include the catheter 220 and pigtail catheter 1100 of FIG. 2a. FIG. 3 shows the base 116 of the distal recurve portion 112 of the pigtail catheter 100 resting within the non-coronary cusp 380. In some embodiments, the base 116 of the distal recurve portion 112 of the pigtail catheter 100 may be advanced into the left coronary cusp or right coronary cusp. In some embodiments, the side port 102 must be on the side of the catheter 100 nearest the space between leaflets of the right coronary cusp and left coronary cusp 370 of the aortic valve. In some embodiments, the pigtail catheter may be twisted and/or adjusted such that the side port 102 is faced toward any one of the leaflets of the aortic valve. The adjustment of the catheter may be done at any point during the procedure.

In some embodiments, the guidewire 110 when advanced through the side port 102, is at the correct angle and distance from the aortic valve to penetrate between the right cusp and left cusp leaflets 370. In some embodiments, the height of the side port 102 relative to the leaflets 370 may be configured to accommodate the unique anatomy of a different patient and or body lumen. In some embodiments the angle of the side port 102 may be configured to direct the guidewire to a different location within a body lumen. The guidewire 110, when advanced through the leaflets of the aortic valve enters the ventricular space 360. The pigtail catheter 100 may then be removed from the non-coronary cusp 380 and aorta 350, leaving the guidewire 110 in place through the aortic valve and into the ventricular space. In some embodiments, a second pigtail catheter may be advanced along the guidewire, into the ventricular space. A transcatheter aortic valve replacement (TAVR) wire and device may then be inserted over the second pigtail device as described in more detail below.

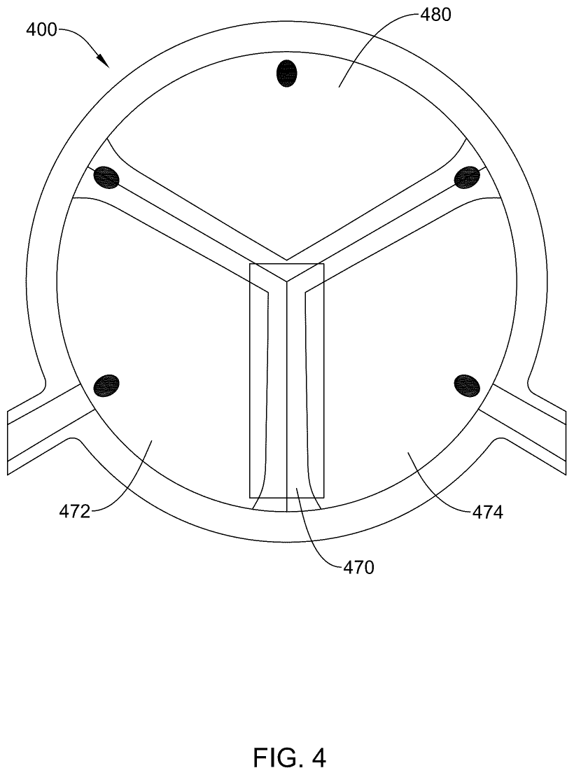

FIG. 4 illustrates the anatomy of a native aortic heart valve 400. Including the non-coronary cusp 480, left coronary cusp 474, and right coronary cusp 472. In some embodiments the non-coronary cusp 480 is the place in which the distal end of a pigtail catheter may rest. The guidewire may exit the pigtail catheter at a controlled angle such that it is directed between the left coronary cusp and right coronary cusp leaflets 470 and into the ventricle. Depending on the disease state of the valve, the pigtail catheter may be placed in the left coronary cusp 474 or the right coronary cusp 472. Depending on calcification, stiffness, or fusing together of leaflets, it may be advantageous to place the pigtail catheter in a certain cusp to target a specific gap. For example, if the left coronary cusp 474 and right coronary cusp 472 have fused together, a physician may decide to advance the pigtail catheter into the right coronary cusp 472, and direct the guidewire between the non-coronary cusp and left coronary cusp leaflets.

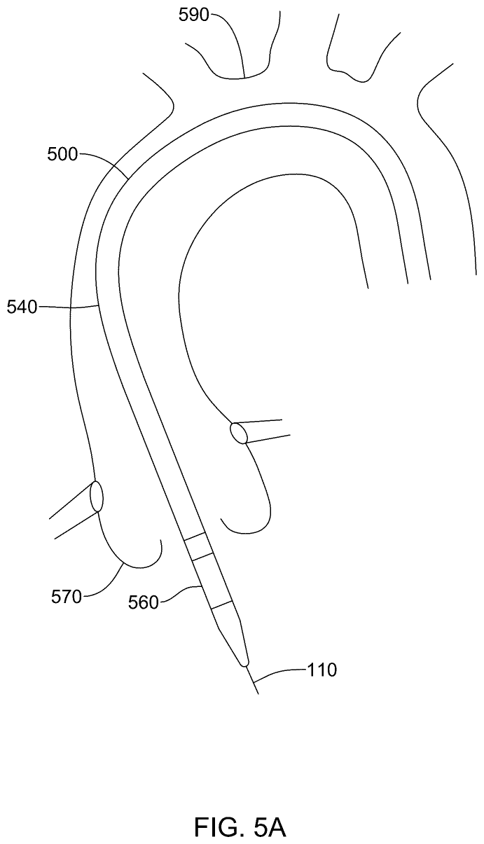

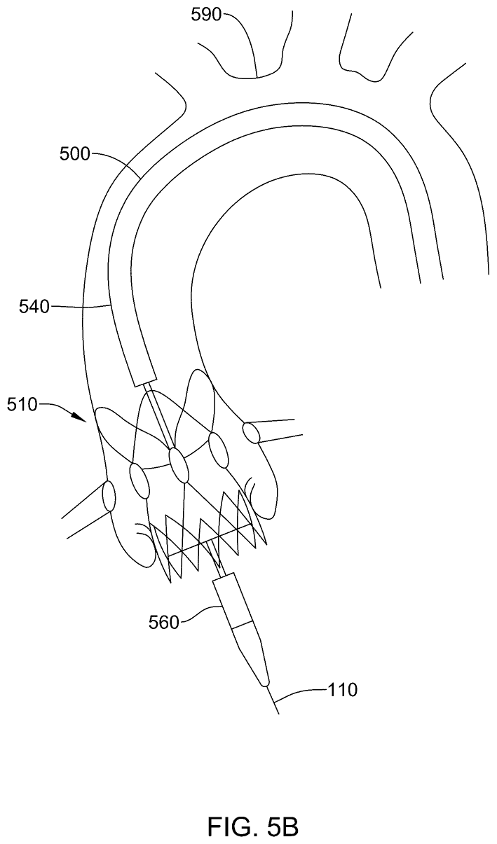

Once the guidewire 110 has been delivered through the aortic valve using the pigtail catheter 100 or catheter 220 described above, a stent-valve delivery system may be advanced over the guidewire, as shown in FIG. 5a. Following the advancement of the guidewire 110 through the aortic valve, a delivery system is advanced along the guidewire to pass through the aortic valve. The delivery system is configured to replace a heart valve. In some embodiments, the delivery system is replacing a native heart valve. In some embodiments the delivery system is replacing a previously implanted heart valve. The delivery system 500 is inserted through a catheter through the femoral artery and vasculature, across the aortic arch 590 and through the aortic valve 570. The delivery system 500 may include a proximal sheath 540 and a distal sheath 560 constraining a replacement stent-valve. The distal sheath 560 may be moved distally and the proximal sheath 540 may be moved proximally to release the stent-valve 510, as shown in FIG. 5B.

It will be understood that the dimensions described in association with the above figure are illustrative only, and that other dimensions and angles of lumens, side ports, and catheters are contemplated. The materials that can be used for the various components of the system for use in replacement heart-valve implant procedures (and/or other systems or components disclosed herein) and the various elements thereof disclosed herein may include those commonly associated with medical devices. For simplicity purposes, the following discussion refers to the system for use in replacement heart-valve implant procedures (and variations, systems or components disclosed herein). However, this is not intended to limit the devices and methods described herein, as the discussion may be applied to other elements, members, components, or devices disclosed herein.

In some embodiments, the system for use in replacement heart-valve implant procedures (and variations, systems or components thereof disclosed herein) may be made from a metal, metal alloy, ceramics, zirconia, polymer (some examples of which are disclosed below), a metal-polymer composite, combinations thereof, and the like, or other suitable material. Some examples of suitable metals and metal alloys include stainless steel, such as 444V, 444L, and 314LV stainless steel; mild steel; nickel-titanium alloy such as linear-clastic and/or super-elastic nitinol; cobalt chromium alloys, titanium and its alloys, alumina, metals with diamond-like coatings (DLC) or titanium nitride coatings, other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R44035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; titanium; platinum; palladium; gold; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-clastic nitinol may be distinguished from super elastic nitinol in that the linear elastic and/or non-super-elastic nitinol does not display a substantial “super-elastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear than the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-clastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. For example, across a broad temperature range, the linear elastic and/or non-super-clastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a super-elastic alloy, for example a super-elastic nitinol can be used to achieve desired properties.

In at least some embodiments, portions or all of the system for use in replacement heart-valve implant procedures (and variations, systems or components thereof disclosed herein) may also be doped with, made of, or otherwise include a radiopaque material. Radiopaque materials are understood to be materials capable of producing a relatively bright image on a fluoroscopy screen or another imaging technique during a medical procedure. This relatively bright image aids a user in determining the location of the system for use in replacement heart-valve implant procedures (and variations, systems or components thereof disclosed herein). Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system for use in replacement heart-valve implant procedures (and variations, systems or components thereof disclosed herein) to achieve the same result.

In some embodiments, the system for use in replacement heart-valve implant procedures (and variations, systems or components thereof disclosed herein) and/or portions thereof, may be made from or include a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex® high-density polyethylene, Marlex® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, polyurethane silicone copolymers (for example, Elast-Eon® from AorTech Biomaterials or ChronoSil® from AdvanSource Biomaterials), biocompatible polymers, other suitable materials, or mixtures, combinations, copolymers thereof, polymer/metal composites, and the like. In some embodiments, the sheath can be blended with a liquid crystal polymer (LCP). For example, the mixture can contain up to about 6 percent LCP.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A system for use in replacement heart-valve implant procedures, the system comprising:
a guidewire; and
an elongate member including a first lumen having a side port disposed proximal of a distal tip of the elongate member;
wherein the guidewire is slideably disposed within the first lumen and configured to be advanced out the side port proximate a leaflet of a native heart valve of a patient;
wherein the side port includes an angled wall defining a distal end of the first lumen;
wherein the angled wall of the side port is configured such that when the guidewire is advanced out of the side port, the guidewire extends out at an angle relative to the elongate member.

2. The system of claim 1, wherein the elongate member is a pigtail catheter.

3. The system of claim 2, wherein the pigtail catheter includes a preformed recurve portion beginning at the distal tip of the pigtail catheter and extending proximally in an unconstrained configuration.

4. The system of claim 3, wherein the preformed recurve portion is sized and shaped to fit within a non-coronary cusp of the patient's valve.

5. The system of claim 1, wherein the angled wall of the side port is configured to direct the guidewire between the right coronary cusp and left coronary cusp of the native heart valve.

6. The system of claim 1, wherein the angled wall of the side port is angled 90 degrees to 120 degrees relative to an inner wall defining the first lumen in a region of the side port.

7. The system of claim 3, wherein the side port is spaced at least 40 mm proximally from a distal end of the preformed recurve portion of the pigtail catheter.

8. The system of claim 1, wherein the elongate member is a catheter that includes the first lumen and a second lumen.

9. The system of claim 8, further comprising a pigtail catheter slideably disposed within the second lumen of the catheter.

10. The system of claim 9, wherein the pigtail catheter includes a preformed recurve portion beginning at a distal tip of the pigtail catheter and extending proximally in an unconstrained configuration.

11. A method of accessing a valve for implanting a replacement heart-valve, the method comprising:
inserting a distal tip of a stent-valve delivery system through a patient's aorta to a position upstream of the aortic valve, the delivery system including:
an elongate member including a first lumen having a side port disposed proximal of a distal tip of the elongate member, and
a guidewire slideably disposed within the first lumen of the elongate member;
advancing the elongate member adjacent the patient's non-coronary cusp;
advancing the guidewire distally out of the side port, the side port including an angled wall defining a distal end of the first lumen, wherein the angled wall of the side port directs a distal end of the guidewire between the right coronary cusp and left coronary cusp of the aortic valve and into a ventricle; and
removing the elongate member, leaving the guidewire extending through the aortic valve.

12. The method of claim 11, wherein the elongate member is a pigtail catheter.

13. The method of claim 12, wherein the pigtail catheter includes a preformed recurve portion beginning at the distal tip of the pigtail catheter and extending proximally in an unconstrained configuration.

14. The method of claim 13, wherein after removing the elongate member the method further comprises inserting a second pigtail catheter over the guidewire and through the aortic valve into the ventricle.

15. The method of claim 14, further comprising, inserting a transcatheter aortic valve replacement device over the second pigtail catheter.

16. The method of claim 15, further comprising removing the second pigtail catheter.

17. A system for use in replacement heart-valve implant procedures, the system comprising:
a pigtail catheter including a side port disposed proximal of a distal tip of the pigtail catheter; and
a guidewire;
wherein the side port has an angled wall configured to direct the guidewire away from the pigtail catheter;
wherein the guidewire is slideably disposed within a first lumen of the pigtail catheter and configured to be advanced out the side port proximate a leaflet of a native heart valve of a patient;
wherein when advanced out of the side port, the guidewire extends out at an angle configured to direct the guidewire through the native heart valve;
wherein the pigtail catheter includes a preformed recurve portion beginning at the distal tip of the pigtail catheter and extending proximally in an unconstrained configuration.

18. The system of claim 17, wherein the side port is disposed proximal of a base of the preformed recurve portion.

19. The system of claim 17, wherein the angled wall of the side port is angled 90 degrees to 120 degrees relative to an inner wall defining the first lumen in a region of the side port.

20. The system of claim 18, wherein the side port is spaced at least 40 mm proximally from the base of the preformed recurve portion of the pigtail catheter.
